(19) United States
(12) Patent Application Publication
(10) Pub. No.: US 2006/0134172 A1
(43) Pub. Date: Jun. 22, 2006

(54) Agents which regulate, inhibit, or modulate the activity and/or expression of lysyl oxidase (LOX) and LOX-like proteases as a unique means to both lower intraocular pressure and treat glaucomatous retinopathies/optic neuropathies

(75) Inventors: Allan Shepard, Fort Worth, TX (US); Abbot F. Clark, Arlington, TX (US); Mark Hellberg, Arlington, TX (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon, Inc.

(21) Appl. No.: 11/293,924

(22) Filed: Dec. 5, 2005

Related U.S. Application Data
(60) Provisional application No. 60/638,090, filed on Dec. 21, 2004.

(51) Int. Cl. A61F 2/00

(52) U.S. Cl. 424/427

(57) ABSTRACT

The present invention provides a method for lowering intraocular pressure and providing neuroprotection to a patient in need thereof by administering a therapeutically effective amount of at least one non-nucleotide or non-protein agent that inhibits expression and/or signaling of lysyl oxidase (LOX) or a lysyl oxidase-like protease (LOXL).

This application claims priority from the provisional application, U.S. patent application Ser. No. 60/638,090 filed Dec. 21, 2004.

### BACKGROUND OF THE INVENTION

1. Field of the Invention

The present invention relates to the field of ocular conditions involving neurodegeneration and/or elevated intraocular pressure. More specifically, the invention provides compositions that lower intraocular pressure and provide ocular neuroprotection.

2. Description of the Related Art

There are a number of ocular conditions that are caused by, or aggravated by, damage to the optic nerve head, degeneration of ocular tissues, and/or elevated intraocular pressure. For example, “glaucomas” are a group of debilitating eye diseases that are a leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Twenty years ago, the interplay of ocular hypertension, ischemia and mechanical distortion of the optic nerve head were heavily debated as the major factors causing progression of visual field loss in glaucoma. Since then, other factors including excitotoxicity, nitric oxide, absence of vital neurotrophic factors, abnormal glial/neuronal is interplay and genomics have been implicated in the degenerative disease process. The consideration of genomics deserves some discussion insofar as it may ultimately define the mechanism of cell death, and provide for discrimination of the various forms of glaucoma. Within the past 8 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congentical glaucoma (CYP1B1), two mapped genes for pigmentary dispersion/pigmentary glaucoma, and a number of genes for developmental or syndromic forms of glaucoma (FOXC1, PITX2, LMX1B, PAX6).

Thus, each form of glaucoma may have a unique pathology and accordingly a different therapeutic approach to the management of the disease may be required. For example, a drug that effects the expression of enzymes that degrade the extracellular matrix of the optic nerve head would not likely prevent RGC death caused by excitotoxicity or neurotrophic factor deficit. In glaucoma, RGC death occurs by a process called apoptosis (programmed cell death). It has been speculated that different types of insults that can cause death may do so by converging on a few common pathways. Targeting downstream at a common pathway is a strategy that may broaden the utility of a drug and increase the probability that it may have utility in the management of different forms of the disease. However, drugs that effect multiple metabolic pathways are more likely to produce undesirable side-effects. With the advent of gene-based diagnostic kits to identify specific forms of glaucoma, selective neuroprotective agents can be tested with the aim of reducing the degree of variation about the measured response.

Glucocorticoids have been associated with the development of ocular hypertension and primary open angle glaucoma (Kass, et al., “Corticosteroid-Induced Glaucoma, In Ritch, R., Shields, M. B., Krupin, T. (eds.),” The Glaucomas, The C. V. Mosby Company, St. Louis, Mo., pp. 1161-1168 (1989); DeSantis, et al., “Dexamethasone-Induction of Ocular Hypertension in the Primate, ARVO Abstracts. Invest. Ophthalmol. Vis. Sci., 31(Suppl.):99 (1990); Knepper, et al., “Intraocular Pressure and Glycosaminoglycan Distribution in the Rabbit Eye: Effect of Age and Dexamethasone,” Exp. Eye Res., 27: 567-575 (1978); Francois, et al., “Ultrastructural and Morphometric Study of Corticosteroid Glaucoma in Rabbits, Ophthalmic Res., 16:168-178 (1984); Lorenzetti, O. J., “Effects of Corticosteroids on Ocular Dynamics in Rabbits,” J. Pharmacol. Exp. Therap., 175:763-772 (1970); and Zhan, et al., “Steroid Glaucoma: Corticosteroid-Induced Ocular Hypertension in Cats,” Exp. Eye Res., 54:211-218 (1992)). Glaucoma patients have also been reported to have higher levels of the endogenous glucocorticoid, cortisol (Rozsival, et al., “Aqueous Humour and Plasma Cortisol Levels in Glaucoma and Cataract Patients,” Current Eye Research, 1:391-396 (1981); Ray, et al., “Plasma Cortisol in Glaucoma,” Ann. Ophthalmol., 9:1151-1154 (1977); and Schwartz, et al., “Increased Plasma Free Cortisol in Ocular Hypertension and Open Angle Glaucoma,” Arch. Ophthalmol., 105:1060-1065 (1987)).

It is known that trabecular meshwork cells have glucocorticoid receptors and that glucocorticoid binding with these receptors causes a change in trabecular meshwork cell gene expression. Known manifestations of this change include a reorganization of the cytoskeleton (Wilson, et al., “Dexamethasone Induced Ultrastructural Changes in Cultured Human Trabecular Meshwork Cells, Cur. Eye Res., 12:783-793 (1993), and Clark, et al., “Glucocorticoid-Induced Formation of Cross-Linked Actin Networks in Cultured Human Trabecular Meshwork Cells,” Invest. Ophthalmol. Vis. Sci., 35:281-294 (1994)) and increased deposition of the extracellular matrix material in trabecular meshwork cells. As a result, the trabecular meshwork becomes “clogged” and unable to perform one of its most critical functions, that is, serving as a gateway for aqueous humor flow from the anterior chamber of the eye. When the aqueous humor flow out of the eye via the trabecular meshwork is diminished, the intraocular pressure of the eye rises. If this state of elevated intraocular pressure is maintained or frequently occurs, the optic nerve head can be damaged resulting in the loss of visual field. Loss of visual field is the hallmark symptom associated with glaucoma.

Current glaucoma therapy is directed to lowering IOP, a major risk factor for the development and progression of glaucoma. These therapies lower IOP, but they do not directly address the pathogenic mechanisms, and the disease continues to progress. Thus, what is needed is a therapeutic method for lowering IOP and/or providing neuroprotection to the optic nerve head and/or to retinal ganglion cells via pathogenic pathways.

### SUMMARY OF THE INVENTION

The present invention overcomes these and other drawbacks of the prior art by providing a method for lowering intraocular pressure and providing neuroprotection to a patient in need thereof by administering a therapeutically effective amount of a composition including at least one non-nucleotide or non-protein agent that inhibits expression and/or signaling of lysyl oxidase (LOX) or the LOX-like proteases LOXL, LOXL2, LOXL3 and LOXL4, and a pharmaceutically acceptable carrier. In another aspect, the invention provides a method for lowering intraocular pressure by administering to a patient a therapeutically effective amount of an agent that inhibits expression and/or signaling of LOX, LOXL, LOXL2, LOXL3 or LOXL4. Preferably, the compositions for use in the method of the invention will lower intraocular pressure that is elevated due to an increased expression of LOX or of a product of LOX signaling.

In preferred embodiments, the composition of the invention may be administered by topical application, intracamerally or via an implant. Typically, the total concentration of the LOX or LOX-like protease inhibitor in the composition of the invention will be from 0.01% to 2%. Generally, the treatment method of the invention will be most useful for a patient suffering from glaucoma, for example normal-tension glaucoma, or ocular hypertension.

The invention further provides a method for preventing the visual field loss associated with POAG by administering to a patient in need thereof a composition including a non-nucleotide or non-protein agent that modulates the expression and/or signaling of LOX or a LOX-like protease such that intraocular pressure is controlled and protection is provided to retinal ganglion cells or to the optic nerve head.

In another embodiment, the present invention provides a composition for lowering intraocular pressure and providing neuroprotection in a patient in need thereof. Generally, the composition of the invention includes at least one agent that inhibits the expression and/or signaling of LOX or a LOX-like protease and a pharmaceutically acceptable carrier. The total concentration of the LOX inhibitor or LOX-like protease inhibitor in the composition of the invention will preferably be from 0.01% to 2%.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. shows the elevation of lysyl oxidase family members in glaucomatous optic nerve head astrocytes.

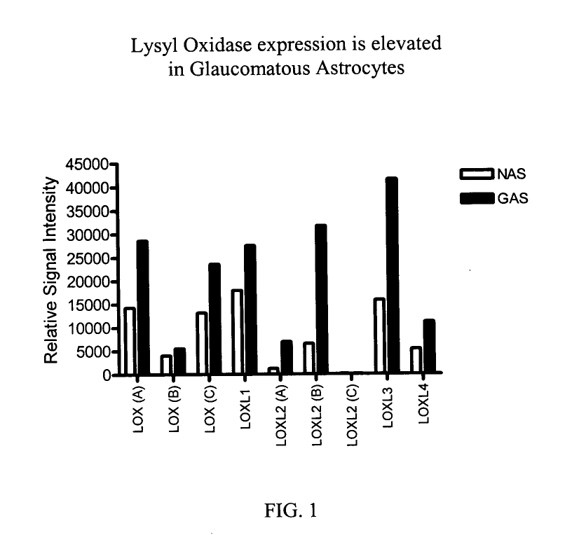

FIG. 2. shows the elevation of lysyl oxidase family members in TGFP2-treated glaucomatous trabecular meshwork TM cells.

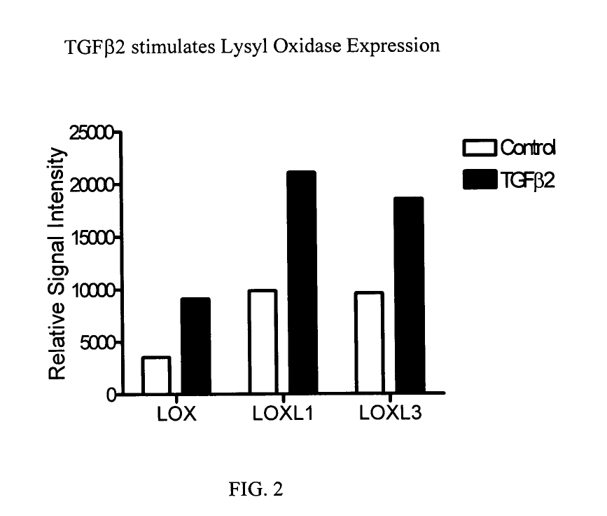

FIG. 3. illustrates the downregulation of lysyl oxidase family members by FOXC1 overexpression in normal and glaucomatous TM cells.

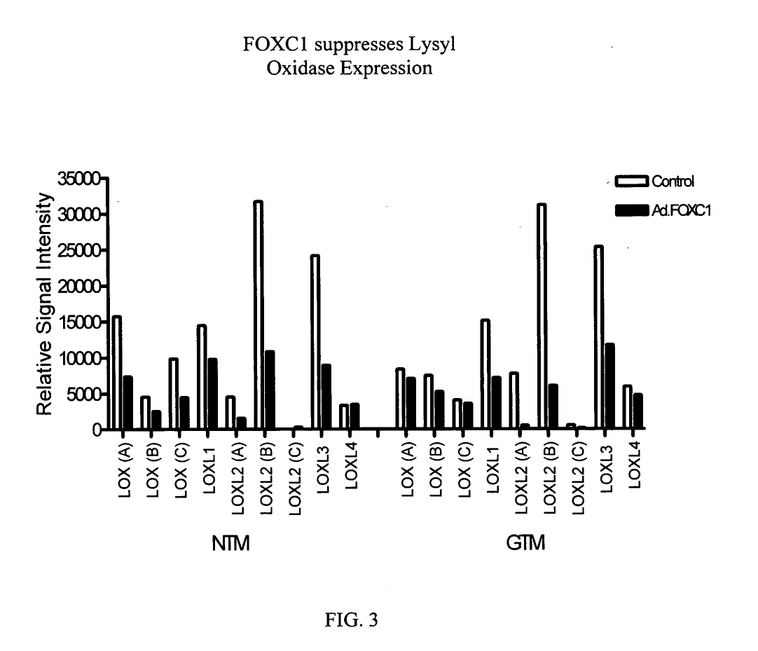

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-comeal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

Lysyl oxidase (LOX) and the LOX-like proteases LOXL, LOXL2, LOXL3 and LOXL4 are extra-cellular copper containing enzymes that initiate the crosslinking of collagens and elastins by catalyzing the oxidative deamination of the ε-amino group in certain lysine and hydroxylysine residues of collagen and lysine residues of elastin. The peptidyl aldehyde formed can condense with other aldehydes and unreacted lysine residues to generate covalent cross-linkages that underlie the insolubility of these connective tissue proteins.

The LOX amine oxidases have a roll in the growth and repair of connective tissue. Increased levels of lysyl oxidase are observed in a variety of fibrotic diseases in which excess collagen is deposited in the affected tissue, such as atherosclerosis, hypertension and pulmonary fibrosis.

The present inventors have discovered that levels of LOX, LOXL (also referred to herein as “LOXL1”), LOXL2, LOXL3, and LOXL4 are elevated in cultured glaucomatous human astrocytes compared to non-glaucoma controls. Optic nerve head astrocytes are thought to be responsible for part of the ECM remodeling seen in glaucoma. When FOXC1 is overexpressed in TM cells, LOX, LOXL1, LOXL2, and LOXL3 gene expression is downregulated. FOXC1 is a transcription factor that causes gene dosage-dependent anterior segment defects, including a disorganized trabecular ECM, in the eye. TGFβ2 treatment of cultured TM cells shows an elevation of LOX, LOXL1, and LOXL3 mRNA levels. TGFβ2 is a growth factor also known to cause changes in ECM molecules when present at supraphysiological levels. Abnormal ECM deposition in the glaucomatous TM is thought to lead to a blockage of outflow and buildup of intraocular pressure (IOP).

Without being bound to theory, it is believed that changes in LOX and LOX-like protease levels in the glaucomatous eye may cause inappropriate cross-linking of collagen and elastin fibers and an imbalance in collagen and elastic tissue homeostasis in the eye. The result may be a change in the stiffness and elasticity of ocular tissue, such as the ONH, TM and sclera, with concomitant IOP elevation and RGC loss consistent with glaucoma pathophysiology.

Thus, in one aspect, the present invention provides a method for lowering IOP and providing neuroprotection to retinal ganglion cells by administering a composition including a non-nucleotide or non-peptidyl LOX inhibitor or LOX-like protease inhibitor. It is further contemplated that the composition could include a compound that inhibits an agent which upregulates LOX or a LOX-like protease inhibitor.

The therapeutic agent for the treatment of glaucoma will preferably be a small drug-like molecule, which affects one or more aspects of the LOX pathway. Preferred therapeutic agents are those that are: (1) inhibitors of LOX or LOX-like proteases; (2) inhibitors of agents acting downstream of LOX action (i.e., inhibitors of LOX signaling) and/or (3) inhibitors of agents that upregulate LOX or LOX-like protease gene or protein expression.

U.S. Pat. No. 4,444,787 discusses the treatment of wounded mammalian ocular tissue to reduce the cross-linking of collage fibrils in the tissue by administering β-aminopropionitrile (BAPN), which is known to cause the in vitro inhibition of lysyl oxidase. The '787 patent seeks to solve the problem of the development of scar tissue due to surgical trauma or trauma to external ocular structures by inhibiting compounds that participate in the collagen cross-linking process. The '787 patent seeks to stabilize the wound healing process at the end of the process, when fibroblasts are no longer metabolically active and manufacturing collagen and evidence of lysyl oxidase activity is minimal. This is due to the fact that inhibiting lysyl oxidase systemically produced undesirable side effects. The '787 patent does not discuss the treatment of glaucoma or lowering IOP by administering inhibitors of LOX or LOX-like proteases.

Agents useful in the methods of the invention include lysyl oxidase inhibitors disclosed in U.S. Pat. Nos. 5,120,764; 4,997,854; 4,943,593; 4,965,288; 5,021,456; 5,182,297; 5,252,608; and 5,059,714; published PCT applications WO02/061092 and WO03/097612; Nagan et al. (Front Biosci 3:A23-A26 (1998)); Liu et al. (J. Biol. Chem. 272:32370-32377 (1997)); Garcheu et al. (J. Biol. Chem. 264:12963-12969 (1989)); Misiorowski et al. (Biochem Biophys Res Commun 85:809-814 (1978)); DiSilvestro et al. (Biochem Pharmacol 32:343-346 (1983)); Wilmarth et al. (J Toxicol Environ Health 37:41-53 (1992)); Sheperd et al. (Biochim Biophys Acta 1647:252-259 (2003)); and Sheperd et al. (Eur J Biochem 269:3645-3658 (2002)) all herein incorporated by reference. Preferred agents for use in the methods of the invention include, but are not limited to, β-aminoproprionitrile, monoamine oxidase inhibitors, tranylcypromine (TCP), homocysteine thiolactone, allenylamines, allylamines, diamines, 2-phenyl-3(2H)-pyridazinones, and C-proteinase inhibitors.

The agents of this invention, can be incorporated into various types of ophthalmic formulations for delivery to the eye (e.g., topically, intracamerally, or via an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art. The is following are examples of possible formulations embodied by this invention.

| (a) Topical ocular formulation | wt. % |
|---|---|
| LOX Inhibitor or LOX-like inhibitor | 0.005-5.0 |
| Tyloxapol | 0.01-0.05 |
| HPMC | 0.5 |
| Benalkonium chloride | 0.01 |
| Sodium chloride | 0.8 |
| Edetate disodium | 0.01 |
| NaOH/HCl | q.s. pH 7.4 |
| Purified water | q.s. 100 mL |

It is further contemplated that the compounds of the invention could be formulated in intraocular insert devices.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

All references cited herein, to the extent that they provide exemplary procedural or other details supplementary to those set forth herein, are specifically incorporated herein by reference.

We claim:

1. A method for lowering intraocular pressure and providing neuroprotection to a patient in need thereof, said method comprising administering a therapeutically effective amount of a composition comprising at least one non-nucleotide or non-protein agent that inhibits expression, signaling or biological functions of lysyl oxidase (LOX), and a pharmaceutically acceptable carrier.

2. The method of claim 1, wherein said administering is by topical application, intracamerally or via an implant.

3. The method of claim 1, wherein the total concentration of said LOX inhibitor in said composition is from 0.01% to 2%.

4. The method of claim 1, wherein said patient suffers from glaucoma or ocular hypertension.

5. The method of claim 5, wherein said glaucoma is normal-tension glaucoma.

6. A method for lowering intraocular pressure in a patient in need thereof, said method comprising administering a therapeutically effective amount of a composition comprising at least one non-nucleotide or non-protein agent that inhibits expression, signaling, or biological functions of lysyl oxidase (LOX), and a pharmaceutically acceptable carrier.

7. The method of claim 7, wherein said administering is by topical application, intracamerally or via an implant.

8. The method of claim 7, wherein the total concentration of said IGF-1 inhibitor in said composition is from 0.01% to 2%.

9. The method of claim 7, wherein said patient suffers from glaucoma or ocular hypertension.

10. The method of claim 11, wherein said glaucoma is normal-tension glaucoma.

11. A method for lowering intraocular pressure in a patient in need thereof, said method comprising administering a therapeutically effective amount of a composition comprising at least one non-nucleotide or non-protein agent that inhibits the expression, signaling, or biological functions of a lysyl oxidase-like (LOXL) protease selected from the group consisting of LOXL1, LOXL2, LOXL3, and LOXL4.

12. A composition for lowering intraocular pressure and providing neuroprotection in a patient in need thereof, said composition comprising at least one agent that inhibits the expression, signaling, or biological functions of lysyl oxidase (LOX) and a pharmaceutically acceptable carrier.

13. The composition of claim 12, wherein the total concentration of said LOX inhibitor in said composition is from 0.01% to 2%.

14. A composition for lowering intraocular pressure and providing neuroprotection in a patient in need thereof, said composition comprising at least one agent that inhibits the expression, signaling, or biological functions of a lysyl oxidase-like protease (LOXL) selected from the group consisting of LOXL1, LOXL2, LOXL3, and LOXL4, and a pharmaceutically acceptable carrier.

15. The composition of claim 14, wherein the total concentration of said LOXL inhibitor in said composition is from 0.01% to 2%.
